(19) United States
(12) Patent Application Publication
(10) Pub. No.: US 2022/0127213 A1
(43) Pub. Date: Apr. 28, 2022

(54) METHOD FOR SELECTIVE SEPARATION, ISOLATION AND RECOVERY OF CANNABIDIOL AND CANNABIDIOL-LIKE MEROTERPENE ACIDS FROM COMPLEX MATRICES

(71) Applicant: Istok NAHTIGAL, Barrys Bay (CA)

(72) Inventor: Istok NAHTIGAL, Barrys Bay (CA)

(21) Appl. No.: 17/428,913

(22) PCT Filed: Feb. 4, 2020
(86) PCT No.: PCT/CA2020/050135

§ 371 Date: Aug. 5, 2021

Related U.S. Application Data
(60) Provisional application No. 62/801,573, filed on Feb. 5, 2019.

(51) Int. Cl. C07C 37/68; B01D 11/02; B01D 9/00

(52) U.S. Cl. CPC C07C 37/685; B01D 9/0054; B01D 11/0288

(57) ABSTRACT

Described is a method of selectively isolating non-rigid structure meroterpenes (for example, cannabidiolic acid) from a complex matrix that may also contain rigid structure meroterpenes (for example, THCa), comprising selectively precipitating the non-rigid structure meroterpenes in the form of a triethylamine salt complex by adding triethylamine; isolating the triethylamine salt complex from the mother liquor; then heating the triethylamine salt complex to vaporize the triethylamine, leaving an isolated neutral non-rigid structure meroterpene. In certain embodiments, the starting product is a cannabis resin that has been solubilized in, for example, d-limonene.

### TECHNICAL FIELD

The invention relates in general to cannabinoids. In particular the invention is related to the selective separation, isolation, purification and recovery of certain cannabinoids from a heterogeneous mixture.

### BACKGROUND OF THE INVENTION

Cannabinoids, or more accurately phytocannabinoids are mostly known for their occurrence in the genus Cannabis, which on a high level encompasses several different chemotaxonomic varietals: i) expressing predominantly THCa with very low levels of CBDa, ii) which is more fibrous and has higher levels of CBDa, iii) an intermediate between the two, iv) with high levels of cannabigerol (CBGa), and v) with negligible levels of cannabinoids. Phytocannabinoids have also been identified in several other plant species. In Cannabis, they are found in the form of their carboxyl derivatives, the cannabinoid carboxylic acids. These can be transformed to “neutral cannabinoids” via a decarboxylation reaction (i.e. elimination of CO₂).

Cannabis is a complex plant, believed to contain over 400 chemical entities; several dozen different cannabinoids have been isolated from the plant. Though concentrated in a resin produced in the glandular trichomes, cannabinoids can also be found in other plant tissue, such as the leaf and stem, albeit in much lower concentration. Cannabis has been used for centuries both for its recreational and therapeutic effects, with different cannabinoids believed to be the main contributors to these effects, by binding to various cannabinoid receptors in the brain and elsewhere in the body. Two different classes of cannabinoid receptors are currently known, termed CB1 and CB2, with CB1 receptors found primarily in the brain and reproductive system and CB2 receptors predominantly found in the immune system and spread out through the peripheral nervous system. Different cannabinoids act on each receptor class differently, defined by its specificity and affinity.

Though different species of plant, and even different parts of the same plant are known to contain different concentrations and ratios of cannabinoids, some of the primary cannabinoids in cannabis are tetrahydrocannabinol (THC; (6aR,10aR)-6,6,9-trimethyl-3-pentyl-6a,7,8,10a-tetrahydro-6H-benzo[c]chromen-1-01; (−)-Δ⁹-THC), cannabidiol (CBD; 2-((1R,6R)-3-methyl-6-(prop-1-en-2-yl)cyclohex-2-enyl)-5-pentylbenzene-1,3-diol); cannabinol (CBN; 6,6,9-trimethyl-3-pentylbenzo[c]chromen-1-01); cannabigerol (CBG; 2-[(2E)-3,7-dimethylocta-2,6-dienyl]-5-pentylbenzene-1,3-diol); tetrahydrocannabivarin (THCV; (6aR,10aR)-6,6,9-trimethyl-3-propyl-6a,7,8,10a-tetrahydrobenzo[c]chromen-1-01); cannabidivarin (CBDV; 2-[(1R,6R)-3-methyl-6-prop-1-en-2-ylcyclohex-2-en-1-yl]-5-propylbenzene-1,3-diol); cannabidiorcinol (CBDO, 5-methyl-2-[(1˜{6}˜{R})-3-methyl-6-prop-1-en-2-ylcyclohex-2-en-1-yl]benzene-1,3-diol) and cannabichromene (CBC; 2-methyl-2-(4-methylpent-3-enyl)-7-pentylchromen-5-ol) and the recently discovered cannabidiphorol (CBDP; 2-((1R,6R)-3-methyl-6-(prop-1-en-2-yl)cyclohex-2-enyl)-5-heptylbenzene-1,3-diol).

Certain of these compounds are found at least in part in their carboxylic acid forms, for example, THC as tetrahydrocannabinolic acid (THCa; (6aR,10aR)-2-carboxy-6,6,9-trimethyl-3-pentyl-6a,7,8,10a-tetrahydrobenzo[c]chromen-1-olate), CBD as cannabidiolic acid (CBDa; 2,4-dihydroxy-3-[(1R,6R)-3-methyl-6-prop-1-en-2-ylcyclohex-2-en-1-yl]-6-pentylbenzoic acid), and CBDV as cannabidivarinic acid (CBDVa; 2,4-dihydroxy-3-[(1R,6R)-3-methyl-6-prop-1-en-2-ylcyclohex-2-en-1-yl]-6-propylbenzoic acid) and CBDP as cannabidiphorolic acid (CBDPa; 2,4-dihydroxy-3-[(1R,6R)-3-methyl-6-prop-1-en-2-ylcyclohex-2-en-1-yl]-6-heptylbenzoic acid).

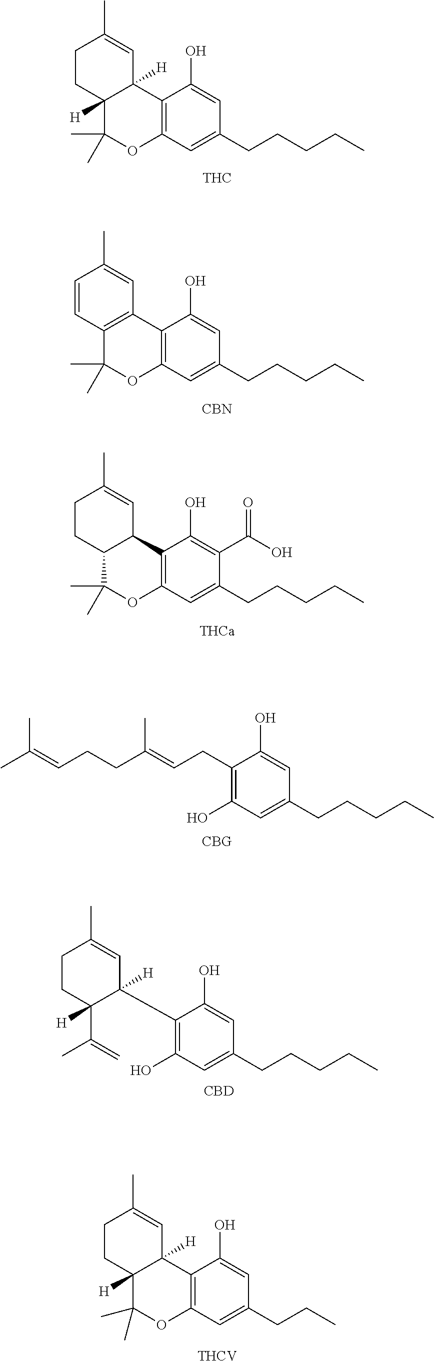

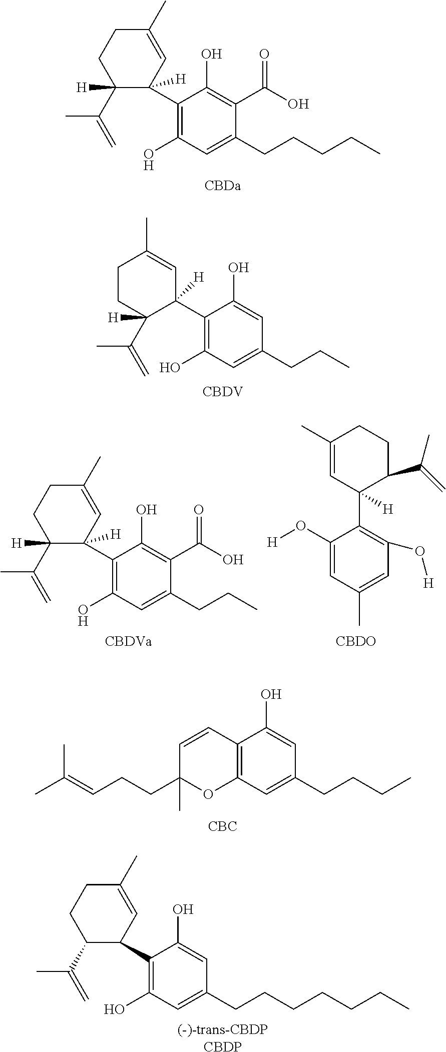

Chemical structures and numbering system for CBD and D9-THC type cannabinoids. CBD, cannabidiol; THC, tetrahydrocannabinol.

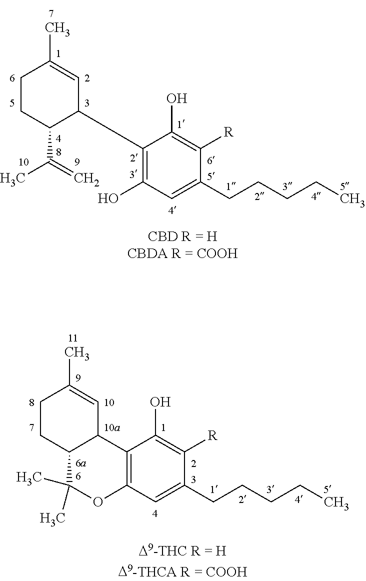

The well-known psychoactive or more specifically intoxicating effects of cannabis are generally associated with THC, and its psychoactive metabolite, 11-OH-THC. THC is known to bind to the CB1 cannabinoid receptors in the brain and central nervous system.

CBD, in contrast, is believed to have no intoxicating effects by its own, though it may attenuate effects of THC. CBD appears to act as an indirect antagonist of cannabinoid agonists, but does not appear to act at the CB1 and CB2 receptors, instead possibly acting as a 5HT1a receptor agonist.

In general, most cannabinoids can be defined in two groups: the rigid structure meroterpene and meroterpene acids (referred to henceforth as rigid meroterpenes), which have two or three rings bonded in a fashion to introduce a rigid structure, and which include THC, THCa, CBN, CBC, CBCa, and THCV to list a few and non-rigid structure meroterpenes and meroterpene acids (referred to henceforth as non-rigid meroterpenes), which have rings or unsaturated carbon chains that may structurally orient to be ring-like of which are freely rotatable through a single bond to an aromatic ring or ring system, and which include CBD, CBDa, CBG, CBGa, CBDV, CBDVa, CBDP and CBDPa. Novel cannabidiol-quinone and hydroxyquinone derivatives (CBD-Q, CBD-HQ derivatives) are taught in WO2015/158381 (incorporated herein by reference), which also fall into the non-rigid structure meroterpenes.

Cannabinoid-based therapeutics are known, with some approved in several countries. Sativex™ (GW Pharmaceuticals, UK) is an aerosolized mist for oral administration containing a near 1:1 ratio of CBD and THC, and is approved in several countries, including Canada, for multiple sclerosis—related and cancer-related pain. Marinol™ (Solvay Pharmaceuticals, Belgium) is a THC-based drug used to treat poor appetite, nausea, and sleep apnea. And most recently Epidolex™ (GW Pharmaceuticals, UK), an FDA approved plant-derived CBD indicated for the treatment of seizures associated with Lennox-Gastaut syndrome (LGS) or Dravet syndrome in patients 2 years of age and older.

Cannabinoids for pharmaceutical use are either separated from a plant source, or manufactured synthetically by either full chemical synthesis, semi-synthetic or via biosynthetic routes. Separation of the cannabinoids from the complex matrix of the plant trichomes is typically done by extraction with organic solvents, such as hydrocarbons and alcohols. Alternatively, supercritical solvent extraction with carbon dioxide can be used. Similar approaches are taken to recover the cannabinoids from non-plant matrices such as fermentation broths, organic overlays and solvents in the case of chemical synthesis. A process for production of cannabinoid carboxylic acid salts from which a mixture of neutral cannabinoids can be obtained is described in US2015/0038567A1, incorporated by reference. However, this process is nonselective in that both rigid and non-rigid meroterpenes are non-selectively obtained.

The process described therein includes solubilization of the plant material in a solvent, for example water-immiscible solvents such as: hydrocarbons with up to 30 carbon atoms, liquefied hydrocarbons gaseous in the normal state such as propane or butane, petroleum distillates such as petroleum ether ligroin, kerosene, naphtha, halogenated hydrocarbons with up to 12 carbon atoms, carbon disulfide, esters and ethers with up to 16 carbon atoms, and mixtures of said solvents; as well as water miscible solvents including water with basic additives such as ammonia, alkylamines, hydroxylamine, hydrazine, metal hydroxides, metal carbonates or metal hydrogen carbonates, water with detergents, lower alcohols with up to 4C atoms, acetonitrile, propionitrile, acetone, and mixtures thereof; also carbon dioxide and liquefied sulfur dioxide, liquefied ammonia and liquefied alkylamines. Specifically, the patent discloses use of alcohols, esters, ethers, ketones, hydrocarbons (aliphatic and/or alicyclic), halogenated hydrocarbons, and nitriles with up to 20 carbon atoms.

Once the cannabinoids have been solubilized, US 2015/0038567A1 teaches that the cannabinoid carboxylic acids in the solution can be precipitated out as crystalline salts with suitable bases. Suitable bases are taught to include dicyclohexylamine, ammonia, alkoxides, hydroxides, carbonates, hydrogen carbonates, carboxylates and other basic salts of elements of the first, second and third main group and of tin, lead and bismuth, and the alkoxides, hydroxides, carbonates, hydrogen carbonates, carboxylates and other basic salts of transition elements such as for example silver. Further suitable organic bases are taught to be pharmaceutical active substances with at least one basic nitrogen atom in the molecule, such as for example morphine, hydromorphone, buprenorphine, etc. The only examples in the patent utilize dissolving the cannabinoids in isopropanol and precipitating in dicyclohexylamine. The resultant precipitated dicyclohexamine salts of cannabinoids are non-specific in that all cannabinoid acids are precipitated out. Separation of THC from CBD, for example, is not taught, and would require further purification and isolation by known methods, for example, chromatography.

Purification of specific cannabinoids, and/or separation of rigid from non-rigid meroterpenes has been a laborious and expensive process, typically requiring chromatographic techniques, due to the structural similarities between the compounds. Purification of specific cannabinoids, specifically, separation of rigid from non-rigid meroterpenes, and for example, separation, isolation and purification of CBD from a mixed source of cannabinoids containing both CBD and THC, is highly desirable, since the compounds have very different pharmacological profiles. (Likewise would a method for separation, isolation and purification of THC from a mixed source containing both CBD and THC.) Obtaining a concentrated CBD extract, powder or crystalized compound, containing a low level of THC, from a complex mixture (ie plant extract resin or fermentation broth), in a manner that avoids the use of chromatography, fractional distillation and/or other expensive or long processes, would be highly desirable. Processes combining reaction and separation into a single, integrated operation are becoming ever more attractive for chemical, pharmaceutical, mining and related industries. Key advantages of reactive separations in comparison to the conventional approaches based on multiple unit operations is that operations combining reaction and separation are integrated into a single unit that allows the simultaneous production, isolation and removal of products. This improves productivity and selectivity, capital cost reduction, reduces the amount of energy and solvent usage, hence waste reduction and leads to high-efficiency systems with built-in “green” chemistry and engineering attributes.

D-limonene is an aliphatic cyclic monoterpene and is the major component in the oil of citrus fruit peels. It is primarily used as a flavoring agent in food manufacturing. D-limonene is considered to be GRAS as a food additive when used as a synthetic flavoring substance and adjuvant.

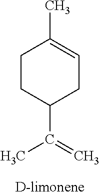

Triethylamine is a tertiary amine commonly used in organic synthesis as a base in the preparation of esters and amides from acyl chlorides. It is primarily used in the production of quaternary ammonium compounds, though it has a number of other uses, including as a Drosophila anesthetic.

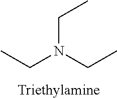

### BRIEF DESCRIPTION OF THE FIGURES

FIG. 1A shows cannabis resin dissolved in D-limonene according to one step in a method of the invention.

FIG. 1B shows selective precipitation of certain cannabinoids by complexation with triethylamine, from a solution of cannabis resin dissolved in D-limonene, according to one step in a method of the invention.

FIG. 2 shows a photograph of an isolated cannabidiolic acid-amine complex made according to a method of the invention.

FIG. 3 shows an HPLC compositional analysis of the cannabidiolic acid-amine complex of FIG. 2.

FIG. 4 shows an HPLC compositional analysis of the cannabidiolic acid-amine complex formed according to another method of the invention.

FIG. 5 shows a photograph of an amorphous CBD resin made by a method of the invention.

FIG. 6 shows a photograph of CBD crystals made by a method of the invention.

FIG. 7 shows an HPLC compositional analysis of the crystals of FIG. 6.

FIG. 8 shows the ATR-FTIR spectrum of the CBDa:trialkylamine crystalline complex.

FIG. 9 shows TGA and DSC thermograms of the CBDa:trialkylamine crystalline complex.

FIG. 10 show the TGA and derivative TGA (DTG) signals quantitatively indicating the rate of complex decomposition-decarboxylation.

### SUMMARY OF THE INVENTION

According to one aspect of the present invention is provided a method of selectively isolating a solid complex comprising one or more non-rigid structure meroterpenes from a complex matrix containing said one or more non-rigid structure meroterpenes, comprising: adding triethylamine to the matrix to precipitate out the solid complex comprising the one or more non-rigid structure meroterpenes, leaving a mother liquor; and removing the mother liquor to obtain the solid complex.

According to a further aspect of the present invention is provided a method of selectively isolating a solid complex comprising one or more non-rigid structure meroterpene from a cannabis plant product, comprising: solubilizing the cannabis plant product in a solvent having a low dielectric constant which is capable of solubilizing cannabinoids, to form a solution containing one or more non-rigid structure meroterpenes; and subjecting the solution containing said one or more non-rigid structure meroterpenes to the above-described method.

In certain embodiments, the solution also comprises a rigid structure meroterpene, which remains in the mother liquor.

In certain embodiments, the solvent is selected from the group consisting of linear hydrocarbons, aliphatic alcohols, esters, and natural solvents, for example, limonene, pinene, and myrcene.

In certain embodiments, the solvent is d-limonene.

In certain embodiments, the cannabis plant product is a cannabis resin.

According to a further aspect of the present invention is provided a method of selectively isolating and purifying a non-rigid structure meroterpene from a solution containing said one or more non-rigid structure meroterpenes, comprising: performing the method as herebefore described on said solution to obtain said solid complex; heating said solid complex at a temperature range of 100-200 degrees Celsius, for example, 120 to 180 degrees Celsius or 140 to 160 degrees Celsius, under vacuum or sweep of inert gas to form an isolated non-rigid structure meroterpene; optionally crystallizing said isolated, non-rigid structure meroterpene.

According to a further aspect of the present invention is provided a method of selectively isolating and purifying a non-rigid structure meroterpene from a cannabis plant product, comprising: solubilizing the cannabis plant product in a solvent having a low dielectric constant which is capable of solubilizing cannabinoids, to form a solution containing one or more non-rigid structure meroterpenes; subjecting the solution containing said one or more non-rigid structure meroterpenes to the method as herebefore described.

In certain embodiments, the cannabis plant product is a cannabis resin.

In certain embodiments, the non-rigid structure meroterpene is cannabidiol.

In certain embodiments, the non-rigid structure meroterpene is cannabidivarol.

In certain embodiments, the rigid structure meroterpene is (−)-Δ⁹-tetrahydrocannabinol.

According to a further aspect of the present invention is provided a method of producing a non-rigid structure meroterpene pharmaceutical product from a cannabis resin, comprising performing the method as herebefore described, and packaging the resultant isolated, non-rigid structure meroterpene in a pharmaceutically acceptable carrier.

In certain embodiments, the non-rigid structure meroterpene pharmaceutical product is a cannabidiol drug.

### DETAILED DESCRIPTION

The present invention solves the growing problem of production of CBD and CBD-like compounds in a simplistic, cost effective manner. The process relies on combination of a specific complexing agent directly with a solubilized mixture containing cannabinoids where a very narrow compound class is selectively precipitated as fine crystals or finely powder, followed by purification by recrystallization if necessary. In certain embodiments, all solvents used in the here described process fall within the Class III solvent classification, making this selection and isolation process extremely attractive for pharmaceutical and NHP (natural health product) applications. The described process contains certain characteristics namely; insensitivity to solvent parameters, high chemical yields and atom economy, regiospecificity and stereospecificity, a large thermodynamic driving force (>20 kcal/mol) to favor a reaction with a single reaction product. Additionally, the process has simple reaction conditions, uses readily available starting materials and reagents, the use of solvent that is benign and easily removed and provides simple product isolation by non-chromatographic methods.

It has been empirically found that one tertiary trialkyl amine, specifically, triethylamine, will selectively complex with non-rigid structure meroterpene acids, over rigid structure meroterpene acids. Through extensive experimentation is was discovered that tertiary trialkyl amines with alkyl moieties with greater than 4 carbons do not lead to appreciable selective separation. Additionally, cyclic tertiary alkyl amines (ex. methenamine) as well as the N-oxide of triethylamine do not lead to this selection. Thus triethylamine, preferably in slight molar excess, can be utilized to precipitate out these non-rigid structure meroterpene acids from a matrix of mixed rigid and non-rigid structure meroterpenes and meroterpene acids. Triethylamine can be used to quickly, easily, cheaply, safely and selectively isolate one or more of, CBDa, CBDVa, CBDOa, CBDPa, or more generally any cannabidiolic acid-like molecule from a solution comprising one or more of these compounds but also comprising one or more of THC, THCa, CBC, CBCa, CBN, THCV and/or other non-listed matrix components. The triethylamine will not measurably precipitate out the rigid structure meroterpenes, or will precipitate out the non-rigid structure meroterpenes so selectively is that it results in a significant concentration of the non-rigid structure meroterpenes (as compared to the rigid structure meroterpenes).

Thus, triethylamine can be used to selectively purify and isolate non-rigid structure meroterpenes from the aforementioned list, from a matrix comprising both non-rigid structure meroterpenes and rigid structure meroterpenes. Illustratively, triethylamine can thus separate CBDa from THCa in a simple, effective, rapid and safe precipitation/complexation step, by adding the triethylamine to the solution and precipitating out the CBDa as a complex.

The solution comprising non-rigid structure meroterpenes and rigid structure meroterpenes can be any such solution, for example, a Cannabis plant product or extract, such as a Cannabis resin which has been solubilized in any known suitable solvent. In certain embodiments, the known suitable solvent has a low dielectric constant and is capable of solubilizing cannabinoids, for example, aliphatic and alicyclic hydrocarbons (C1 to C18), alcohols, simple esters, complex esters such as mono, di and triglyceride oils, natural solvents like limonene (for example, D-limonene) or the pinenes. D-limonene and pinenes (α, β) have the added advantage that they are generally recognized as safe (GRAS), and work particularly well. It is noted that the method would work equally well for isolating and/or purifying non-rigid structure meroterpenes from a solution that does not contain rigid structure meroterpenes, for example, a solution of (bio)synthetically produced CBDa, containing a complex mixture of impurities, the selection advantage of the invention can again be utilized.

Thus, in one embodiment, an organic extraction solution comprising triethylamine was contacted with a D-limonene solubilized cannabinoid resin, whereby the CBDa, CBDVa were precipitated out of solution leaving a depleted mother liquor. The precipitate was recovered as a finely divided crystalline salt and washed. The crystalline salt was subsequently used to isolate a purified neutral CBD isolate by thermal dissociation of the complexing amine, with concurrent decarboxylation yielding the freed cannabinoid acid. The trialkylamine was recovered by condensation, the liberated carbon dioxide removed via vacuum, leaving the neutral counterpart. Alternatively, un-complexed CBDa can be recovered from the crystalline salt by a substitution reaction, wherein the amine complexing agent is displaced by a ligand with greater affinity.

General Considerations

HPLC analyses were recorded in an Agilent 1100 HPLC system equipped with a vacuum degasser, quaternary pump and autosampler with a DAD detector. System was equipped with a Restek Raptor ARC-18 4.6 mm×150 mm, 2.7 μm column. The sample at the appropriate dilution was dissolved in ethanol and injected (5 μL) for analysis.

Thermogravimetric analysis (TGA-DSC) was recorded in a thermogravimetric analyzer, TA Instruments SDT Q600. The sample (14.84 mg of complex) was weighed into a 100 μL alumina crucible and sealed with a lid. Samples were heated at 2° C./min from 20 to 500° C., under a nitrogen flow of 60 mL/min.

ATR-FTIR data was acquired on Nicolet FTIR 6700 Fourier transform infrared spectrophotometer equipped with an Ever-Glo mid/far IR source, a potassium bromide beam splitter, and a deuterated triglycine sulfate detector.

General Example

According to one general embodiment, purified CBD was obtained from Cannabis sativa as follows.

Resin was extracted from Cannabis sativa plant using liquid carbon dioxide (CO₂) and subsequently solubilized using a suitable solvent. In certain embodiments, the solvent volume was reduced to concentrate, simplifying handling during the complexation step. In certain embodiments, the resultant solubilized cannabis resin can have a CBDa concentration spanning 1 to 60% (w/w).

Complexing amine solution (comprising triethylamine) in a quantity equivalent to, or in excess of, the molar concentration of acidic cannabinoids (or alternatively, but not ideally, less than the molar concentration of acidic cannabinoids) was added to the solubilized resin with stirring. Upon addition of the complexing amine agent, instantaneous precipitation of the meroterpene acid complex was observed, resulting in a precipitated microcrystalline slurry. The complexing reaction was efficient at room temperature; similar results were observed both at lower and elevated temperatures. However, it was found that it was preferable that the complexing occur at below 60 degrees Celsius, to avoid initiating decarboxylation of the acid cannabinoid.

The precipitated microcrystalline slurry of complexed CBDa:triethylamine salt was pressure filtered through a 5 μm pore size filter, and the filtrate was retained for analysis and post processing. The complexed CBDa:triethylamine salt was washed several times with neat solvent. The complexed CBDa:triethylamine salt was then again vacuum filtered and allowed to dry at ambient temperature under a gentle stream of inert gas, for example nitrogen or argon. After drying, in certain embodiments, the complex was recrystallized from a suitable solvent, to further increase the purity by removal of trapped reaction solvent in the initial precipitated microcrystals.

Alternatively, the method can also be used to purify rigid structure meroterpenes—by precipitating out the non-rigid structure meroterpenes, then obtaining the rigid structure meroterpenes from the mother liquor. For example, the mother liquor can be decanted (post-precipitation with triethylamine) and the rigid structure meroterpenes can be precipitated by addition of alternative higher molecular weight tertiary amines. As well, the method can be used to purify a solution containing non-rigid structure meroterpenes regardless of whether rigid structure meroterpenes are present—resulting in an excellent and efficient method for purifying, for example, a synthetically prepared CBDa in solution with its synthesis impurities.

### Example 1: Method for Production of CBDa:Triethylamine Salt from Cannabis Resin

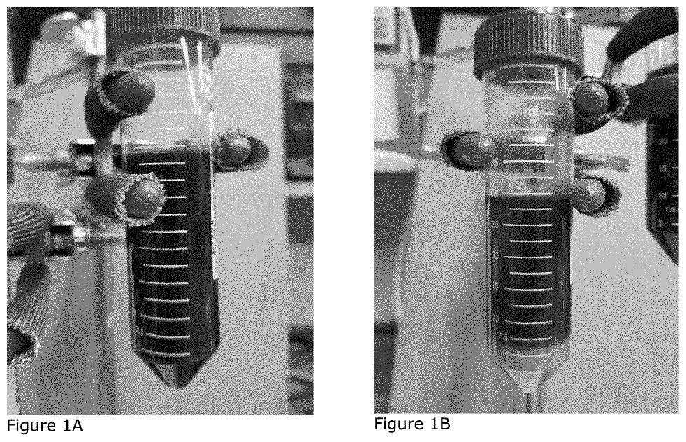

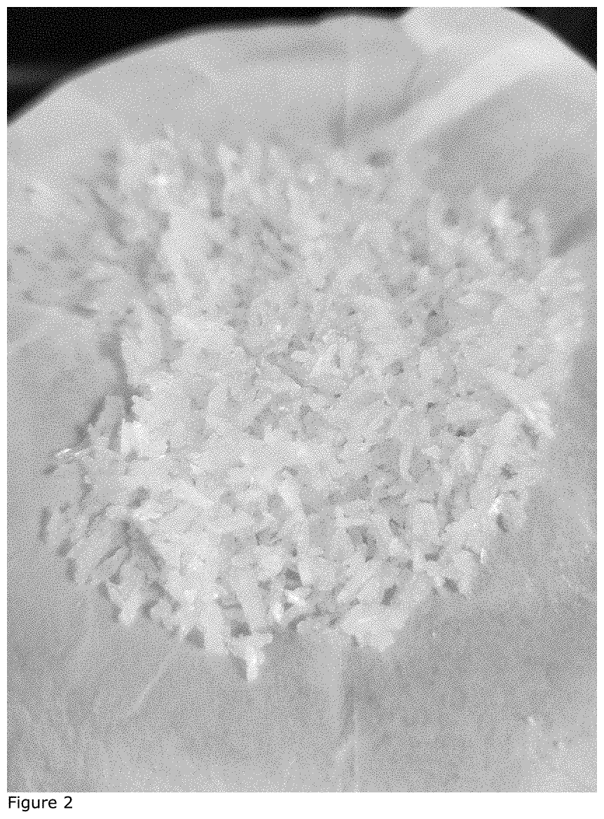

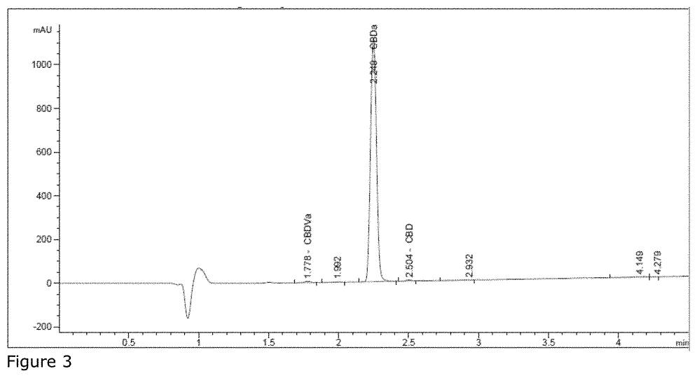

5 g of CO₂—extracted cannabis resin was dissolved in 25 ml of d-limonene (shown dissolved in FIG. 1A). The resultant solution contained 34% CBDa, 11.5% THCa, as well as neutral cannabinoids in lower concentrations, as determined by HPLC analysis of the dissolved resin. 2 mL (a molar excess) of triethylamine was added to the solution, with stirring, at room temperature. A dense white precipitate of CBDa:triethylamine salt was nearly instantly formed (shown in FIG. 1B). The white precipitate was filtered and washed three times with neat d-limonene, then dried, resulting in the obtaining of 2.2 g of a CBDa:triethylamine salt with a purity of 96.7%, with a minor presence of CBDVa:triethylamine. A photograph of the resultant CBDa:triethylamine salt is shown in FIG. 2; an HPLC analysis of the salt is shown in FIG. 3.

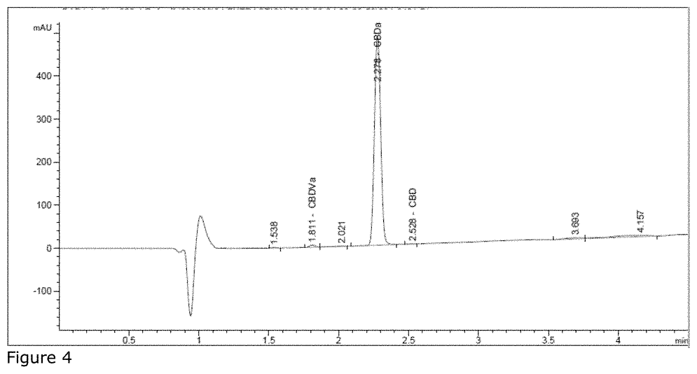

Interestingly, comparative examples utilizing a solvent with higher dielectric constant (i.e. ethyl acetate, isopropanol) resulted in an incomplete precipitation of the CBDa and CBD from the solubilization media, with much more of the aforementioned remaining in the mother liquor solution. Thus, though solubilization was quite effective, it resulted in a much lower yield of the precipitated CBDa:triethylamine salt. When ethyl acetate was used as the solubilization media, intermediate yields were produced. It appeared that d-limonene was therefore a better solvent media than either [polar solvent] or ethyl acetate for the method. However, all yielded pure, selective, CBDa:triethylamine salt, albeit of different yields (see, for example, FIG. 4, which shows an HPLC analysis of the salt formed after solubilization with ethyl acetate followed by precipitation with triethylamine).

Also interestingly, precipitation with other, similar, tertiary trialkyl amines, such as methenamine, tributylamine, and trimethylamine, was found to be less or entirely non-specific for non-rigid structure meroterpenes, with both non-rigid structure, and rigid structure meroterpenes precipitating out of solution.

### Example 2: Method for Production of CBDa:Triethylamine Salt from Cannabis Resin

15 g of limonene-extracted cannabis resin was dissolved in 150 ml of d-limonene. The resultant solution contained 11.2% CBDa, 7.5% THCa, as well as neutral cannabinoids in lower concentrations, as determined by HPLC analysis. 2 mL (a molar excess) of triethylamine was added to the solution, with stirring, at room temperature. A heavy white precipitate of CBDa:triethylamine salt was nearly instantly formed. The white precipitate was resuspended, filtered and washed three times with neat d-limonene, then dried, resulting in the obtaining of 2.4 g of a CBDa:triethylamine salt with a purity of 96.7%.

### Example 3: Thermal Decomposition-Decarboxylation of Cannabinoid Acid Amine Salt

The CBDa:triethylamine salt from Examples 1 or 2 was heated to molten state at the gram scale; melting began at 145 degrees Celsius, and was held at 155 degrees Celsius, under vacuum for one trial and a sweep of Argon gas for the other. The heating caused the triethylamine portion of the salt to be evolved as a vapor, which could be separately condensed and recovered for reuse if desired. The heating also caused carbon dioxide to be liberated from the cannabinoid acid through decarboxylation. Upon cooling, the neutral cannabinoid exhibited as a clear, highly viscous liquid with the gradual purple tinting of the CBD melt attributed to the formation of the CBD hydroxyquinone.

### Example 4: Formation of Pure, Crystalized CBD

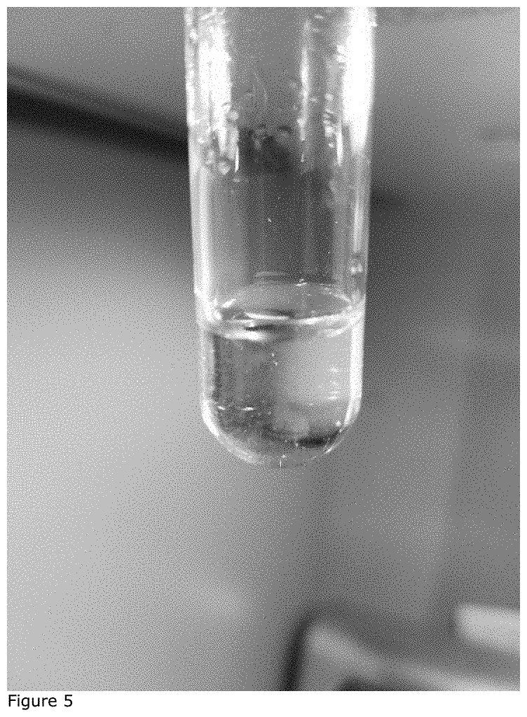

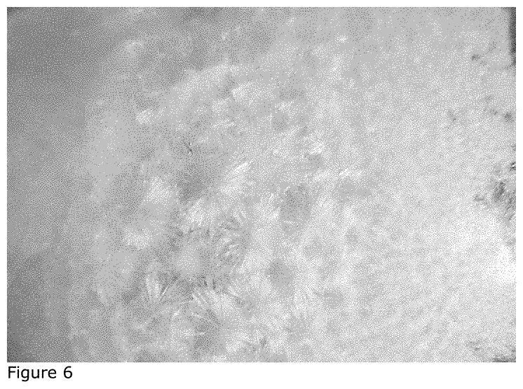

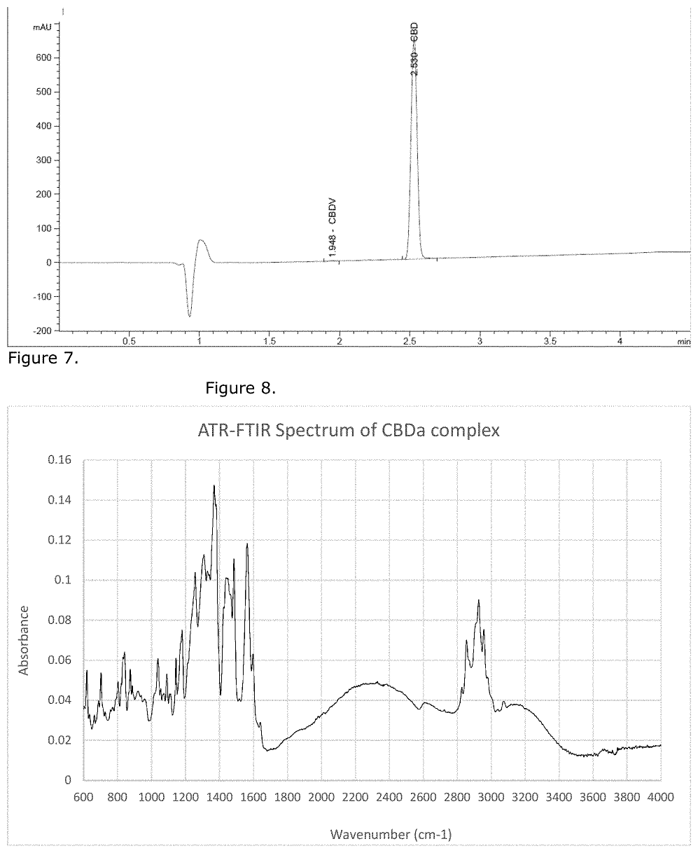

The liquid resultant from Example 3 was allowed to cool under vacuum, leaving a semi-liquid CBD resin (FIG. 5). This resin was induced to crystallize, by introduction of a small CBD seed crystal and yielded 3.2 g of CBD crystal having a purity of 99.7%. Photographs of the resultant crystals were shown in FIG. 6, with their HPLC analysis shown in FIG. 7. To note, amorphous solidification of the melt will occur spontaneously on its own with a delayed onset.

### Example 5: FTIR Spectra and TGA-DSC Thermograms of Crystalline CBDa Complex

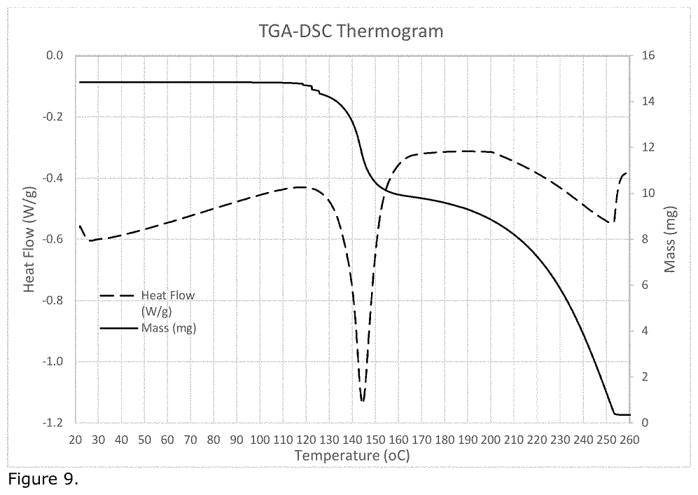

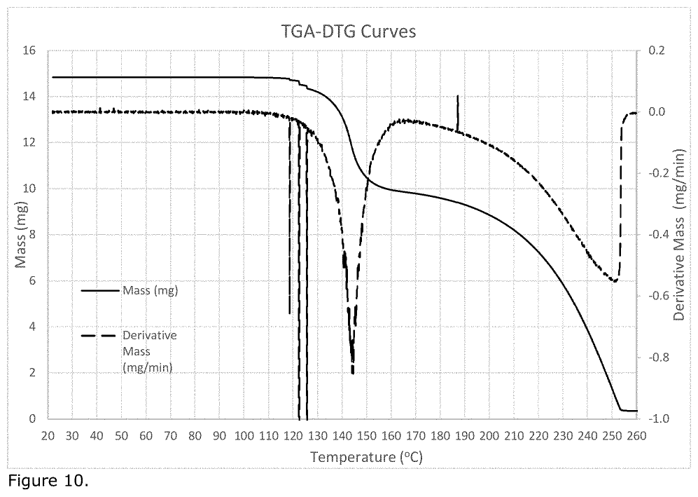

The meroterpene complex of the present invention was further characterized by recording the FTIR spectrum (FIG. 8). This methodology can be used as a means of identification and fingerprinting. TGA-DSC analysis is referenced in FIG. 9. The TGA-DSC analysis of the complex showed an endothermic event with its maximum at 144.5° C.; a second event to note occurring between 160-200° C., where the heat flow is constant spanning the heating of the melt. DTG analysis (FIG. 10.) showed the highest rate of 0.85 mg/min occurring at a temperature of 144.5° C. complimenting the endothermic event characterized as the simultaneous decomposition-decarboxylation of the CBDa:NEt₃ complex into gaseous CO₂, NEt₃ and molten neutral CBD.

1. A method of selectively isolating a solid complex comprising one or more non-rigid structure meroterpenes from a solution containing said one or more non-rigid structure meroterpenes, comprising:
(a) adding triethylamine to the solution to precipitate out the solid complex comprising the one or more non-rigid structure meroterpenes, leaving a mother liquor;
(b) removing the mother liquor to obtain the solid complex.

2. A method of selectively isolating a solid complex comprising one or more non-rigid structure meroterpene from a cannabis plant product, comprising:
(i) solubilizing the cannabis plant product in a solvent having a low dielectric constant which is capable of solubilizing cannabinoids, to form a solution containing one or more non-rigid structure meroterpenes;
(ii) subjecting the solution containing said one or more non-rigid structure meroterpenes to the method of claim 1.

3. The method of claim 1 wherein the solution also comprises a rigid structure meroterpene, which remains in the mother liquor.

4. The method of claim 2 wherein the solvent is selected from the group consisting of linear hydrocarbons, aliphatic alcohols, esters, and natural solvents.

5. The method of claim 4 wherein the natural solvent is selected from the group consisting of limonene, pinene, and myrcene.

6. The method of claim 4 wherein the solvent is d-limonene.

7. The method of claim 2 wherein the cannabis plant product is a cannabis resin.

8. A method of selectively isolating and purifying a non-rigid structure meroterpene from a solution containing said one or more non-rigid structure meroterpenes, comprising:
(A) Performing the method of claim 1 on said solution to obtain said solid complex;
(B) Heating said solid complex at a temperature range of 120 to 170 degrees Celsius under vacuum or sweep of inert gas to form an isolated non-rigid structure meroterpene;
(C) Optionally crystallizing said isolated, non-rigid structure meroterpene.

9. A method of selectively isolating and purifying a non-rigid structure meroterpene from a cannabis plant product, comprising:
(I) Solubilizing the cannabis plant product in a solvent having a low dielectric constant which is capable of solubilizing cannabinoids, to form a solution containing one or more non-rigid structure meroterpenes;
(II) Subjecting the solution containing said one or more non-rigid structure meroterpenes to the method of claim 8.

10. The method of claim 9 wherein the cannabis plant product is a cannabis resin, fermentation broth or overlay or chemical process solvent.

11. The method of claim 1 wherein the non-rigid structure meroterpene is cannabidiol.

12. The method of claim 1 wherein the non-rigid structure meroterpene is cannabidivarol.

13. The method of claim 1 wherein the non-rigid structure meroterpene is cannabidiphorol.

14. The method of claim 1 wherein the non-rigid structure meroterpene contains alkyl chain length in the range of C1 to C16 in position 5 on the resorcinyl moiety.

15. The method of claim 1 wherein the rigid structure meroterpene is (−)-Δ⁹-tetrahydrocannabinol.

16. A method of producing a non-rigid structure meroterpene pharmaceutical product from a cannabis resin, comprising performing the method of claim 9 and packaging the resultant isolated, non-rigid structure meroterpene in a pharmaceutically acceptable carrier.

17. The method of claim 16 wherein the non-rigid structure meroterpene pharmaceutical product is a cannabidiol drug.
